(19) United States
(12) Patent Application Publication
(10) Pub. No.: US 2023/0368092 A1
(43) Pub. Date: Nov. 16, 2023

(54) DYNAMIC CALENDAR SCHEDULING BASED UPON USER DATA

(71) Applicant: Lenovo (United States) Inc., Morrisvile, NC (US)

(72) Inventors: Matthew Fardig, Boonville, IN (US); Mark Delaney, Raleigh, NC (US)

(21) Appl. No.: 17/742,835

(22) Filed: May 12, 2022

(51) Int. Cl. G06Q 10/06; G06Q 10/10; A61B 5/16; A61B 5/00

(52) U.S. Cl. CPC G06Q 10/06312; G06Q 10/1095; A61B 5/165; A61B 5/486

(57) ABSTRACT

One embodiment provides a method, the method including: receiving, at a dynamic scheduling system, biometric data of a user captured by at least one sensor; determining, using the dynamic scheduling system, the biometric data indicates a characteristic of the user is outside a range; and modifying, using the dynamic scheduling system and based upon the determining, at least one event of a calendar of the user. Other aspects are described and claimed.

### BACKGROUND

An increase in remote working has also seen an increase in meetings, particularly virtual meetings. Since workers are not congregated in a single location where they can see other workers and communicate with the workers, one option is to schedule a meeting to discuss or communicate with other workers. Additionally, since workers are not at the same location physically, it can be hard to identify that someone has another meeting except by looking at a calendar of the user. However, many meetings are pop-up meetings where the meeting is not scheduled a significant time in advance, but rather a few minutes or possibly an hour in advance of the meeting. Additionally, since workers commonly work together within a group or with a few individuals on a reoccurring basis, these workers may have standing reoccurring meetings. Thus, it can be difficult to ensure that a worker does not become overloaded with meetings.

### BRIEF SUMMARY

In summary, one aspect provides a method, the method including: receiving, at a dynamic scheduling system, biometric data of a user captured by at least one sensor; determining, using the dynamic scheduling system, the biometric data indicates a characteristic of the user is outside a range; and modifying, using the dynamic scheduling system and based upon the determining, at least one event of a calendar of the user.

Another aspect provides an information handling device, the information handling device including: at least one sensor; a processor operatively coupled to the at least one sensor; a memory device that stores instructions that, when executed by the processor, causes the information handling device to: receive, at a dynamic scheduling system, biometric data of a user captured by the at least one sensor; determine, using the dynamic scheduling system, the biometric data indicates a characteristic of the user is outside a range; and modify, using the dynamic scheduling system and based upon the determining, at least one event of a calendar of the user.

A further aspect provides a product, the product including: a computer-readable storage device that stores executable code that, when executed by a processor, causes the product to: receive, at a dynamic scheduling system, biometric data of a user captured by at least one sensor, using the dynamic scheduling system, the biometric data indicates a characteristic of the user is outside a range; and modify, using the dynamic scheduling system and based upon the determining, at least one event of a calendar of the user.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example of information handling device circuitry.

FIG. 2 illustrates another example of information handling device circuitry.

FIG. 3 illustrates an example method for dynamically modifying a calendar of a user based upon biometric data of the user.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

Workers may become overloaded with meetings and find it difficult to actually accomplish work except by working more hours than planned. This causes workers to become burnt out with work and may even lead to the worker finding a different job. In addition to becoming overloaded with meetings, workers may be involved in meetings that cause negative feelings like frustration, anger, stress, confusion, and/or the like. When the worker has these negative feelings and immediately must engage in another meeting, these negative feelings may not only carry over into the new meeting, but may also become worse. If this pattern continues for an extended period of time, the worker becomes upset at the job. Not only does the inability to perform any work other than participate in meetings affect productivity, but the negative feelings also affect productivity. Thus, reducing meetings for a worker is helpful.

Conventional techniques for modifying calendars all rely on information already contained within the calendar. One conventional solution is to analyze the calendar and identify trends within the calendar to reschedule meetings that are already included on the calendar. These conventional solutions may also provide reminders to users to take breaks or to schedule breaks. The conventional solutions may also provide a timer when a meeting is about to end or may send a reminder to end a meeting early to allow for a break after the meeting. However, all of these solutions rely on information already contained within the calendar and do not take into account any information of the user, for example, the emotional state of the user.

Accordingly, the described system and method provides a technique for dynamically modifying a calendar of a user based upon biometric data of the user. The dynamic scheduling system receives biometric data of a user captured by at least one sensor. The biometric data may be captured while the user is interacting with another user, for example, while the user is in a meeting with another user, while a user is communicating with another user, and/or the like. The biometric data may also be captured while the user is performing a task, for example, preparing for a meeting, working on a presentation, working on work-related matters, and/or the like. It should be noted that while the discussion focuses on work-related matters, the disclosure is not so limited as it can be applied to personal matters.

The biometric data may include pulse information, heart rate, pupil dilation, perspiration, blood pressure, and/or the like. The biometric data may be a specific measurement of different health metrics or other information of a user. From the biometric data, the system can determine if a characteristic of the user is outside a range. The characteristic may be a correlation of the biometric data to a state or context of the user, for example, an emotional state of the user, an environmental context of the user, and/or the like. If the characteristic is outside the range, the system modifies at least one event of a calendar of the user. In other words, as the system receives information regarding biometric data of the user, the system can dynamically modify the calendar of the user.

Therefore, a system provides a technical improvement over traditional methods for calendar scheduling. The described meeting can dynamically adjust or modify a calendar of a user based upon information of the user currently being captured. Thus, instead of relying on information contained in the user's calendar as with conventional systems, the described system it able to integrate currently captured user information with the calendar information. Using the currently captured user information, the system is able to dynamically change the calendar of the user in order to assist in reducing negative feelings of the user. While conventional systems may provide reminders to take breaks, these conventional systems do not take any action to actually make sure that the break is allowed. The described system, on the other hand, can take steps to dynamically modify the calendar of the user so that breaks can be taken when needed. Thus, the described system provides a system that is able to provide a better mental state for a user as compared to conventional systems.

The illustrated example embodiments will be best understood by reference to the figures. The following description is intended only by way of example, and simply illustrates certain example embodiments.

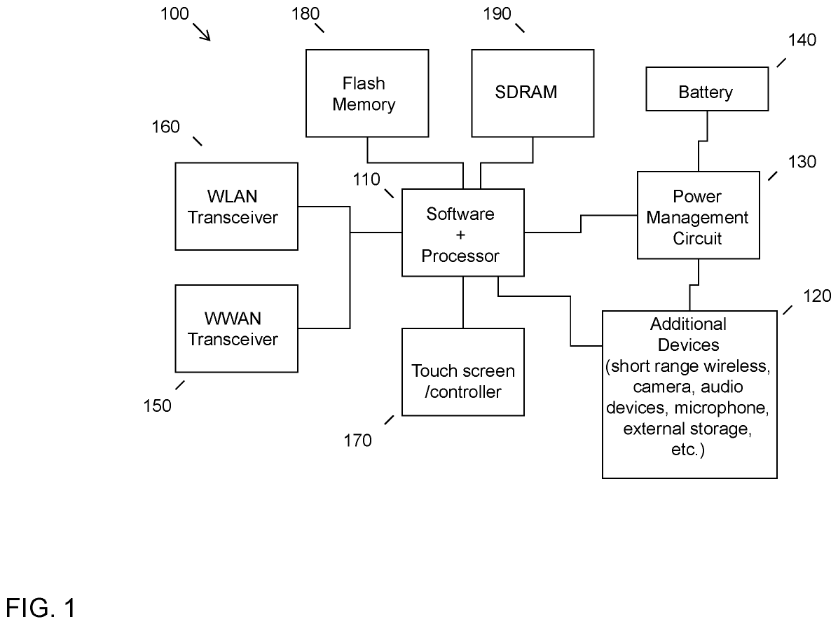

While various other circuits, circuitry or components may be utilized in information handling devices, with regard to smart phone and/or tablet circuitry 100, an example illustrated in FIG. 1 includes a system on a chip design found for example in tablet or other mobile computing platforms. Software and processor(s) are combined in a single chip 110. Processors comprise internal arithmetic units, registers, cache memory, busses, input/output (I/O) ports, etc., as is well known in the art. Internal busses and the like depend on different vendors, but essentially all the peripheral devices (120) may attach to a single chip 110. The circuitry 100 combines the processor, memory control, and I/O controller hub all into a single chip 110. Also, systems 100 of this type do not typically use serial advanced technology attachment (SATA) or peripheral component interconnect (PCI) or low pin count (LPC). Common interfaces, for example, include secure digital input/output (SDIO) and inter-integrated circuit (I2C).

There are power management chip(s) 130, e.g., a battery management unit, BMU, which manage power as supplied, for example, via a rechargeable battery 140, which may be recharged by a connection to a power source (not shown). In at least one design, a single chip, such as 110, is used to supply basic input/output system (BIOS) like functionality and dynamic random-access memory (DRAM) memory.

System 100 typically includes one or more of a wireless wide area network (WWAN) transceiver 150 and a wireless local area network (WLAN) transceiver 160 for connecting to various networks, such as telecommunications networks and wireless Internet devices, e.g., access points. Additionally, devices 120 are commonly included, e.g., a wireless communication device, external storage, etc. System 100 often includes a touch screen 170 for data input and display/rendering. System 100 also typically includes various memory devices, for example flash memory 180 and synchronous dynamic random-access memory (SDRAM) 190.

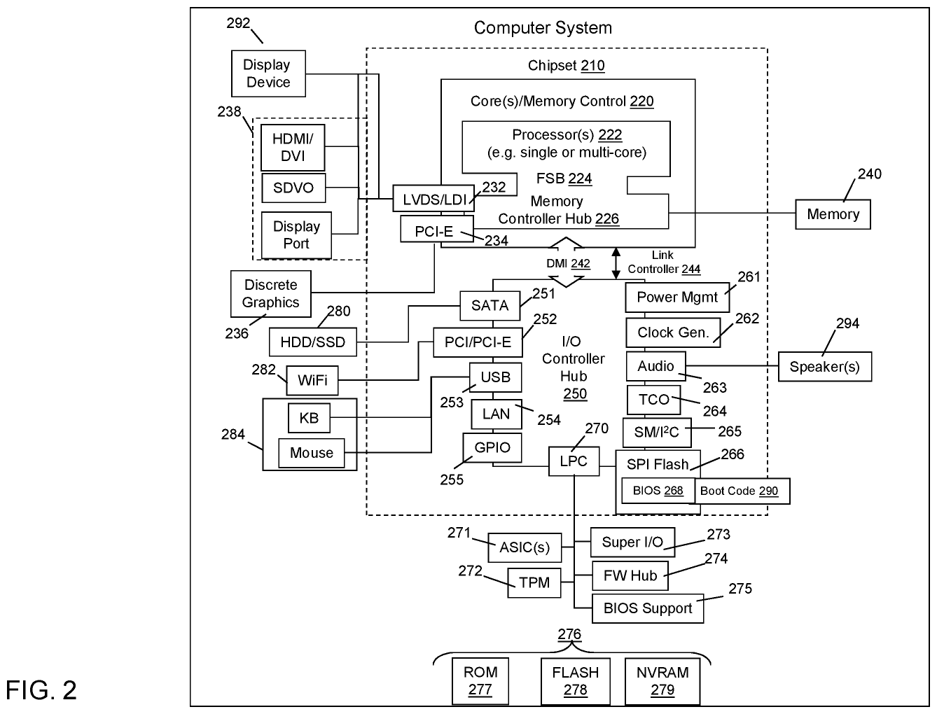

FIG. 2 depicts a block diagram of another example of information handling device circuits, circuitry or components. The example depicted in FIG. 2 may correspond to computing systems such as personal computers, or other devices. As is apparent from the description herein, embodiments may include other features or only some of the features of the example illustrated in FIG. 2.

The example of FIG. 2 includes a so-called chipset 210 (a group of integrated circuits, or chips, that work together, chipsets) with an architecture that may vary depending on manufacturer. The architecture of the chipset 210 includes a core and memory control group 220 and an I/O controller hub 250 that exchanges information (for example, data, signals, commands, etc.) via a direct management interface (DMI) 242 or a link controller 244. In FIG. 2, the DMI 242 is a chip-to-chip interface (sometimes referred to as being a link between a “northbridge” and a “southbridge”). The core and memory control group 220 include one or more processors 222 (for example, single or multi-core) and a memory controller hub 226 that exchange information via a front side bus (FSB) 224; noting that components of the group 220 may be integrated in a chip that supplants the conventional “northbridge” style architecture. One or more processors 222 comprise internal arithmetic units, registers, cache memory, busses, I/O ports, etc., as is well known in the art.

In FIG. 2, the memory controller hub 226 interfaces with memory 240 (for example, to provide support for a type of random-access memory (RAM) that may be referred to as “system memory” or “memory”). The memory controller hub 226 further includes a low voltage differential signaling (LVDS) interface 232 for a display device 292 (for example, a cathode-ray tube (CRT), a flat panel, touch screen, etc.). A block 238 includes some technologies that may be supported via the low-voltage differential signaling (LVDS) interface 232 (for example, serial digital video, high-definition multimedia interface/digital visual interface (HDMI/DVI), display port). The memory controller hub 226 also includes a PCI-express interface (PCI-E) 234 that may support discrete graphics 236.

In FIG. 2, the I/O hub controller 250 includes a SATA interface 251 (for example, for hard-disc drives (HDDs), solid-state drives (SSDs), etc., 280), a PCI-E interface 252 (for example, for wireless connections 282), a universal serial bus (USB) interface 253 (for example, for devices 284 such as a digitizer, keyboard, mice, cameras, phones, microphones, storage, other connected devices, etc.), a network interface 254 (for example, local area network (LAN)), a general purpose I/O (GPIO) interface 255, a LPC interface 270 (for application-specific integrated circuit (ASICs) 271, a trusted platform module (TPM) 272, a super I/O 273, a firmware hub 274, BIOS support 275 as well as various types of memory 276 such as read-only memory (ROM) 277, Flash 278, and non-volatile RAM (NVRAM) 279), a power management interface 261, a clock generator interface 262, an audio interface 263 (for example, for speakers 294), a time controlled operations (TCO) interface 264, a system management bus interface 265, and serial peripheral interface (SPI) Flash 266, which can include BIOS 268 and boot code 290. The I/O hub controller 250 may include gigabit Ethernet support.

The system, upon power on, may be configured to execute boot code 290 for the BIOS 268, as stored within the SPI Flash 266, and thereafter processes data under the control of one or more operating systems and application software (for example, stored in system memory 240). An operating system may be stored in any of a variety of locations and accessed, for example, according to instructions of the BIOS 268. As described herein, a device may include fewer or more features than shown in the system of FIG. 2.

Information handling device circuitry, as for example outlined in FIG. 1 or FIG. 2, may be used in devices such as tablets, smart phones, personal computer devices generally, and/or electronic devices, which may be used in calendar systems and/or biometric data systems. For example, the circuitry outlined in FIG. 1 may be implemented in a tablet or smart phone embodiment, whereas the circuitry outlined in FIG. 2 may be implemented in a personal computer embodiment.

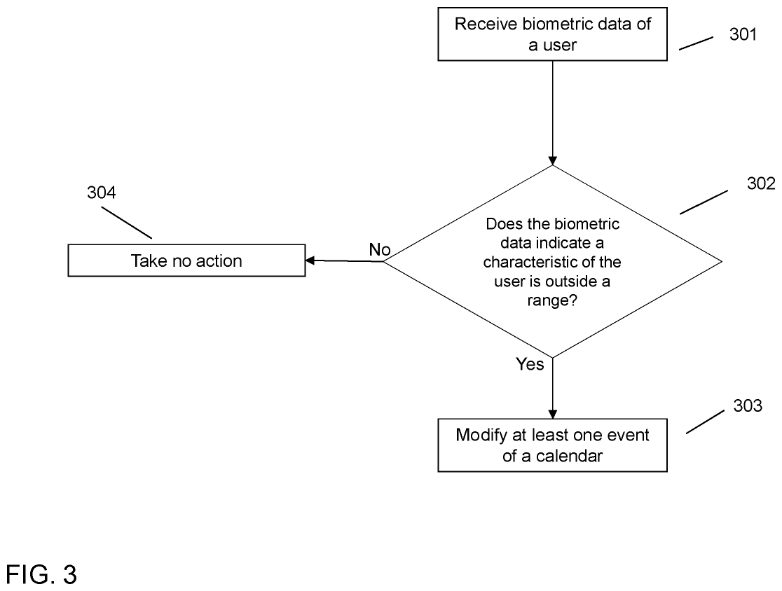

FIG. 3 illustrates an example method for dynamically modifying a calendar of a user based upon biometric data of the user. The method may be implemented on a system which includes a processor, memory device, output devices (e.g., display device, printer, etc.), input devices (e.g., keyboard, touch screen, mouse, microphones, sensors, biometric scanners, etc.), image capture devices, and/or other components, for example, those discussed in connection with FIG. 1 and/or FIG. 2. While the system may include known hardware and software components and/or hardware and software components developed in the future, the system itself is specifically programmed to perform the functions as described herein to dynamically modify a calendar of a user. Additionally, the dynamic scheduling system includes modules and features that are unique to the described system.

The dynamic scheduling system may be a stand-alone application that communicates with other applications, for example, calendar applications, applications having calendar features, and/or the like, to identify calendar events and make any modifications to calendar events. The stand-alone application may be installed on each individual device that utilizes the dynamic scheduling system or may be installed on a network device (e.g., local network device, remote network device, cloud network device, etc.) and accessed by each device utilizing the dynamic scheduling system. Alternatively, the dynamic scheduling system may be an add-on application or feature of existing applications, for example, those applications noted above. For example, the dynamic scheduling system can be provided as an update to an application, thereby providing the dynamic calendaring functions described herein.

At 301, the dynamic scheduling system receives biometric data of a user captured by at least one sensor. Biometric data may be measurement of a metric of the user, for example, a measurement of a health metric of the user, a measurement of a movement metric of a user, and/or the like. A health metric may include a blood pressure measurement, pupil dilation, perspiration, heart rate, breathing rate, oxygen level, facial feature locations, and/or the like. A movement metric may include, but is not limited to, an elevation of the user, an acceleration of the user, a specific location of a user, and/or the like. Accordingly, some example sensors that may be utilized include, but are not limited to, gyroscopes, pulse oximeters, perspiration meters, image capture devices, audio capture devices, heart rate monitors, activity trackers, accelerometers, global positioning system devices, or any other sensors that can be used to capture biometric data, and/or the like. The sensors can include be located on a single device, across devices within an environment of the user, a combination of multiple sensors on a single device and sensors from other devices, and/or the like. The biometric data is any data that can be correlated to a characteristic of the user that is used to determine how a user is feeling and whether the user is developing or experiencing negative, positive, or other types of feelings which may indicate that a modification to the calendar should be made.

The biometric data can be associated with a particular user. Thus, the dynamic scheduling system can mark the biometric data as corresponding to a particular user. Additionally, the dynamic scheduling system can store the biometric data and associate the stored biometric data with the user. This stored data can then be used as historical data to identify trends of the user, provide comparison data, and/or the like. Since the biometric data is associated with a particular user, the biometric data can be stored in an off-site data storage location with other biometric data of other users. Additionally, or alternatively, the biometric data can be stored locally on an information handling device of the user.

The biometric data is captured by the sensor(s) while the user is performing a task, for example, interacting with at least one other user, working on a work matter, working on a personal matter, and/or the like. Thus, the biometric data is captured in real-time as a user is performing a task. The biometric data is then transmitted to the dynamic scheduling system for analysis by the dynamic scheduling system. In the case that the task is an interaction with another user, the interaction may be occurring utilizing a communication medium because the two or more users are not physically co-located, for example, a virtual meeting, a phone call, instant message, and/or other communication medium. Additionally, or alternatively, the interaction, or a portion of the interaction, may be occurring without a communication medium because at least two of the users are physically co-located, for example, in a meeting room, at an office cubicle or other space, and/or the like.

The interaction may also include a combination of communication mediums, a combination of communication mediums and non-communication mediums, and/or the like. For example, some users may connect to a virtual meeting using a personal computer, while other users may connect to the same meeting using a voice call, for example, by dialing in using a telephone. As another example, some users may connect to a meeting virtually through an information handling device, and some users participating in the meeting may be physically co-located, for example, within a meeting room, at a user's workspace, in a public location, and/or the like.

The task may also be something that does not involve interacting with another user. For example, the user may be working on a work-related matter. While the user is working on the work-related matter, sensors can capture the biometric data of the user and transmit the biometric data to the system. As another example, the user may be addressing a personal matter. While the user is addressing the personal matter, sensors can capture the biometric data of the user and transmit the biometric data to the dynamic scheduling system for analysis at 302.

At 302, the dynamic scheduling system determines if the biometric data indicates a characteristic of the user is outside a range. The characteristic may be a state of the user, a context of the user, and/or the like. Thus, the biometric data can be correlated to a particular state, context, and/or the like, of the user. The state of a user may be an emotional state of the user, an activity state of the user (e.g., moving, still, intermittent movement, etc.), a facial expression state of the user, and/or the like. A context may be a particular activity of the user (e.g., sitting, standing, pacing, fidgeting, moving up and down, etc.), an environment of the user, and/or the like. Thus, the system can make a characteristic classification from the biometric data. The classification of the characteristic includes determining the state and/or context of the user, which indicates whether a user is in a good state of mind for additional interactions with other users. For example, from the biometric data, the system may determine that a user is stressed, relaxed, angry, overwhelmed, pacing, has gone to a different location from a typical working location to calm down, calm, and/or the like.

To make the correlation of the biometric data to the characteristic, the dynamic scheduling system may analyze the biometric data and compare the biometric data against setpoint biometric data. The setpoint biometric data may indicate biometric data values that correspond to different characteristics of a user. To identify a characteristic of a user, the system may utilize multiple biometric data values, for example, biometric data values from different biometric data types. As an example, the system may utilize a heart rate, breathing rate, pupil dilation, and oxygen level to determine a characteristic of a user. Different characteristics may need a different number of biometric data types to identify the characteristic.

The system may also attempt to identify a characteristic with a single biometric data value, and some characteristics may only need a single biometric data value, and then assign a confidence level to the identified characteristic. Low confidence levels indicate that the system is leaning towards a particular characteristic classification, but is not confident. Additional biometric data values may then be used to increase the confidence level. The system may then make a determination regarding the indication of the characteristic being outside a range once the confidence level has reached a particular threshold. If additional biometric data values are unavailable, the system may make a determination regarding the characteristic being outside a range, may continue to monitor the biometric data values to make more accurate determinations, or may provide an indication to a user that the confidence level is below a particular threshold. The user may then provide input indicating whether the determination is accurate. The system can then use this information to further refine the determination classification to make the system more accurate and increase a confidence level for subsequent determinations.

The dynamic scheduling system may also utilize a machine-learning model or other learning algorithm to make a determination of whether a characteristic is outside a range from the biometric data. The machine-learning model or other learning algorithm can be trained on historical biometric data and characteristic classifications. The trained model can then be presented with the biometric data and make predictions regarding the characteristic classification. As predictions are made, feedback is automatically ingested by the model to further refine the model and make the model more accurate. Since the biometric data is associated with a user, the machine learning model or other learning algorithm is unique to the user.

Once a characteristic is classified, the system can determine if the characteristic is outside a range. The range may be a preferred state and/or context of the user. For example, the preferred state may be calm, happy, relaxed, and/or the like. Similarly, the preferred state may be a typical working location of the user, the user performing an activity as usual, and/or the like. Thus, the range may not be a standard number range, but rather a feeling, context, and/or the like. Outside the range may be a state and/or context that is not the preferred state and/or context. For example, if the preferred state is calm and the user is agitated, this may be flagged outside the range. Thus, the range is unique to the user and is based upon learned correlations between biometric data

As should be understood, when discussing feelings of a user, feelings may fall anywhere within a range of feelings or emotions. For example, while the preferred state may be calm, there are many states between calm and angry. Thus, the system may determine how far the state is outside the preferred range and, if the state is within a particular set of states including the preferred state, the system may identify the state as being within the range. As an example, the system may identify that happy, calm, and relaxed and all are acceptable states. Thus, the system may be programmed with a set of preferred states and/or contexts, may include a state chart that identifies a linear mapping of degrees of states and/or contexts, and/or the like.

In addition to making a correlation between the biometric data and a state and/or context of the user, the system can also make correlations or associations between the biometric data and an event and/or task of the user. The event and/or task may be a calendar event, for example, a meeting or other interaction with another user, an assigned task, a matter being tracked in a time tracking system, and/or the like, that is scheduled and/or assigned for the time that the biometric data is being received. The system can then identify if particular events and/or tasks cause a characteristic of the user to extend beyond the range. The system may also identify characteristics of the event, for example, participants, event time, event day, frequency of the event, and/or the like, and determine correlations between attributes of an event and/or a task and characteristics of the user. For example, the system may determine that a particular meeting that occurs every week causes the user to become frustrated. As another example, the system may determine that a particular participant causes the user to become overwhelmed. Thus, not only may the range be based upon learned correlations between biometric data, but may also be based upon events of the user.

If the dynamic scheduling system determines the biometric data indicates a characteristic of the user is not outside a range at 302, the system may take no action at 304. The system may also continue to monitor biometric data values of the user and iteratively make the characteristic classification determination. As long as the characteristic remains within the range, the system may continue to take no action with respect to the calendar of the user.

If, on the other hand, the dynamic scheduling system determines the biometric data indicates a characteristic of the user is outside a range at 302, the dynamic scheduling system will modify at least one event of a calendar of the user at 303. The modification occurs dynamically and in substantially real-time as the biometric data is being received and the biometric data is indicating the characteristic of the user is outside the range. In other words, the modification occurs as the interaction with the other user is occurring and if the system determines that the user is experiencing a state and/or context that is outside the preferred range. Thus, substantially real-time refers to while the interaction is occurring and the determination regarding the characteristic classification is being made. Dynamically means that the dynamic scheduling system can make modifications while the interaction is occurring and can make modifications based upon the characteristic classification determination.

When modifying the calendar, the system may identify an event that is in temporal or time proximity to the current task (e.g., interaction, work-related matter, personal matter, etc.) that is causing the characteristic of the user to extend beyond the preferred range. Temporal proximity generally refers to an event that occurs at least in the same day as the current task. However, the system may first start with events that are in closest temporal proximity to and occurring after the current task. The system is attempting to modify events that may be most affected by the undesired characteristic of the user or that may more adversely affect the characteristic of the user. Stated differently, the system is attempting to identify events that may be detrimental for the user to attend while currently experiencing an unpreferred characteristic. Additionally, the system is attempting to identify events that can be rescheduled and that would allow a user a break when the user is experiencing the characteristic outside the range.

When the system identifies an event that is a candidate for modification, the system may modify the event, for example, by rescheduling the event to a different day and/or time. Additionally, for some events it may be possible to completely cancel the event, so the modification may include rescheduling the event and/or cancelling the event. To determine if an event can be modified, the system may identify attributes of the event, for example, other participants, a frequency of the event, and/or any of the attributes of events as previously identified. From the attributes the system may identify an importance of the event, an importance of the participants, a number of the participants, a frequency of the event, and/or the like. Events that may be identified as not possible to be rescheduled for a completely different time or date may be events that are identified as important, events that have specific participants, and/or the like. If these events cannot be completely rescheduled, the system may attempt to at least push the event back to allow for a small break for the user before the beginning of the event, for example, pushing the event back by ten minutes. This may not be possible depending on the event attributes, but may be attempted by the system.

If the system determines that a modification can be made, meaning an event can be rescheduled and/or cancelled, the system may automatically make the modification to the calendar, including sending out any updated event invitations as needed. Additionally, since the system can make correlations between tasks or events and the characteristic of the user, the system can identify tasks or events that may cause the characteristic of the user to move outside the range. In identifying the events, the system may make correlations between task attributes and the characteristic. The system can then use these task attributes that have been identified or correlated to causing the characteristic to be outside the range, to identify other events, for example, future events, that may cause the characteristic to be outside the range. For example, the system may identify that a future event has an attribute that is similar to the current task of the user (e.g., same or similar participant list, same event title, same event time and day of the week, same presentation, same work document, etc.).

Once these events are identified, the system can take action with respect to the calendar of the user with respect to these events. For example, the system may block off time in temporal proximity to the future event and/or task, for example, after the future event and/or task, so as to allow the user a break before beginning another event. The amount of time that is blocked may be based upon a monitoring of a user once a characteristic being outside a range is detected. The system may monitor the user to determine how long it takes the characteristic to return to be within the range. This length of time may then be used as the amount of time that is blocked. Different amounts of time may be determined, for example, events and/or tasks having different attributes may appear to have correlations to lengths of time to return the characteristic to within the range, different degrees of the characteristic having correlations to lengths of time to return the characteristic to within the range, and/or the like.

While modifications may occur automatically by the system, the system may also request the user to confirm that a modification should be made. The system may also provide a notification to a user that a modification has been or is able to be made and allow the user to override the modification. The request or notification may not occur with all events. Rather, the system may identify some events that should have user input regarding a modification and some events that do not require user input. For example, the user may provide a list of events, event participants, and/or the like, that require user input before making a modification. As another example, the system may learn events or event attributes that need user input before making a modification.

The system may also learn that some events and/or tasks change the characteristic of the user back towards the desired range and, if one of these events and/or tasks is scheduled after an event causing the characteristic to move outside the range, the system may not modify the event. These events may be identified based upon event and/or task attributes, monitoring the characteristic of the user during the event and/or task, and identifying correlations therebetween. For example, the system may identify that a particular event participant, when in an event with the user and the event does not include other participants, may cause the user to become calm or relaxed. Thus, if such an event is scheduled after the event and/or task causing the characteristic to move outside the range, the system may not modify the event.

The system may also perform additional actions in addition to modifying the event. The system may continue to monitor the biometric data of the user subsequent to modifying the calendar of the user. From the additional monitoring of the biometric data, the system can determine whether the characteristic is changing to be within the desired range. In other words, the system can determine if the user state and/or context is changing and returning to a preferred state and/or context. If the characteristic appears to be returning to the preferred range, the system may take no additional action, including not modifying any additional events on the calendar. If, on the other hand, the characteristic is not changing or is continuing to move further from the range, the system may take additional actions, for example, continuing to modify other events on the calendar of the user.

Other examples of additional actions that can occur in addition to the modification of the calendar or if the system needs to take additional action due to the fact the characteristic is not returning to the preferred range are possible. For example, the system may notify the user that the system is detecting the user is experiencing a characteristic outside the preferred range. The system may also send a prompt to the user to take steps to reduce the characteristic. As another example, the system may take actions to force the user to take a break, for example, preventing access to applications on an information handling device, putting a device to sleep, preventing the user from using communication applications, and/or the like. These are merely example additional actions that can be taken and other additional actions are also contemplated and can be taken by the system.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device that are executed by a processor. A storage device may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random-access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a storage device is not a signal and is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Additionally, the term “non-transitory” includes all media except signal media.

Program code embodied on a storage medium may be transmitted using any appropriate medium, including but not limited to wireless, wireline, optical fiber cable, radio frequency, et cetera, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and program products according to various example embodiments. It will be understood that the actions and functionality may be implemented at least in part by program instructions. These program instructions may be provided to a processor of a device, a special purpose information handling device, or other programmable data processing device to produce a machine, such that the instructions, which execute via a processor of the device implement the functions/acts specified.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized as appropriate, as the explicit illustrated examples are used only for descriptive purposes and are not to be construed as limiting.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method, the method comprising:
receiving, at a dynamic scheduling system, biometric data of a user captured by at least one sensor;
determining, using the dynamic scheduling system, the biometric data indicates a characteristic of the user is outside a range; and
modifying, using the dynamic scheduling system and based upon the determining, at least one event of a calendar of the user.

2. The method of claim 1, wherein the at least one event comprises at least one meeting of a user scheduled within a time proximity of the receiving biometric data.

3. The method of claim 1, comprising associating the biometric data with an event included on a calendar of the user and identifying attributes of the event.

4. The method of claim 3, wherein the modifying at least one event comprises identifying a correlation between at least one of the attributes of the event and the biometric data.

5. The method of claim 4, wherein the modifying at least one event comprises blocking a time in proximity to another occurrence of an event having at least one attribute similar to the at least one of the attributes of the event.

6. The method of claim 1, wherein the range is unique to the user and wherein the range is identified based upon learned correlations between biometric data and events of the user.

7. The method of claim 1, further comprising providing a prompt to the user to reduce the characteristic.

8. The method of claim 1, further comprising monitoring biometric data of the user subsequent to performing the modifying and determining whether the characteristic is changing to be within the range.

9. The method of claim 1, wherein the modifying at least one event occurs dynamically and in substantially real-time as the biometric data is indicating a characteristic of the user is outside a range.

10. The method of claim 1, wherein the biometric data comprises a health metric of the user and wherein the characteristic corresponds to an emotional state of the user.

11. An information handling device, the information handling device comprising:
at least one sensor;
a processor operatively coupled to the at least one sensor;
a memory device that stores instructions that, when executed by the processor, causes the information handling device to:
receive, at a dynamic scheduling system, biometric data of a user captured by at least one sensor;
determine, using the dynamic scheduling system, the biometric data indicates a characteristic of the user is outside a range; and
modify, using the dynamic scheduling system and based upon the determining, at least one event of a calendar of the user.

12. The information handling device of claim 11, wherein the at least one event comprises at least one meeting of a user scheduled within a time proximity of the receipt of the biometric data.

13. The information handling device of claim 11, comprising associating the biometric data with an event included on a calendar of the user and identifying attributes of the event.

14. The information handling device of claim 13, wherein the modifying at least one event comprises identifying a correlation between at least one of the attributes of the event and the biometric data.

15. The information handling device of claim 14, wherein the modifying at least one event comprises blocking a time in proximity to another occurrence of an event having at least one attribute similar to the at least one of the attributes of the event.

16. The information handling device of claim 11, wherein the range is unique to the user and wherein the range is identified based upon learned correlations between biometric data and events of the user.

17. The information handling device of claim 11, further comprising providing a prompt to the user to reduce the characteristic.

18. The information handling device of claim 11, further comprising monitoring biometric data of the user subsequent to performing the modifying and determining whether the characteristic is changing to be within the range.

19. The information handling device of claim 11, wherein the modifying at least one event occurs dynamically and in substantially real-time as the biometric data is indicating a characteristic of the user is above a threshold.

20. A product, the product comprising:
a computer-readable storage device that stores executable code that, when executed by a processor, causes the product to:
receive, at a dynamic scheduling system, biometric data of a user captured by the at least one sensor;
determine, using the dynamic scheduling system, the biometric data indicates a characteristic of the user is outside a range; and
modify, using the dynamic scheduling system and based upon the determining, at least one event of a calendar of the user.
